(19) United States
(12) Patent Application Publication
(10) Pub. No.: US 2023/0075060 A1
(43) Pub. Date: Mar. 9, 2023

(54) MVP SCORE: A LOW PASS WHOLE GENOME SEQUENCING-BASED TEST IN DIFFERENTIATION BETWEEN MULTIPLE PRIMARY LUNG CANCERS AND INTRA-PULMONARY METASTASES

(71) Applicant: The Chinese University of Hong Kong, Hong Kong (CN)

(72) Inventors: Ka Fai TO, Hong Kong (CN); Cheong Kin Ronald CHAN, Hong Kong (CN); Sze Hang Calvin NG, Hong Kong (CN); Hung Man Joanna TONG, Hong Kong (CN); Chit CHOW, Hong Kong (CN); Wai YEUNG, Hong Kong (CN)

(21) Appl. No.: 17/823,373

(22) Filed: Aug. 30, 2022

Related U.S. Application Data
(60) Provisional application No. 63/260,775, filed on Aug. 31, 2021.

(51) Int. Cl. G16B 20/10; C12Q 1/6886; C12Q 1/6809

(52) U.S. Cl. CPC G16B 20/10; C12Q 1/6886; C12Q 1/6809; C12Q 2600/156; C12Q 2600/112

(57) ABSTRACT

The subject invention pertains to methods and systems for the quantification of clonality between tissue samples obtained from a subject having or suspected to have cancer and treatment of the subject tailored to the origin of tumors in the tissue samples as primary cancer or metastases based on the clonality quantification.

### CROSS-REFERENCE TO RELATED APPLICATION

This application claims the benefit of U.S. Patent Application Ser. No. 63/260,775, filed Aug. 31, 2021, which is hereby incorporated by reference in its entirety including any tables, figures, or drawings.

### BACKGROUND OF THE INVENTION

Non-small cell lung cancer (NSCLC) is a leading cause of cancer-related deaths worldwide. According to World Health Organization, lung cancer globally accounted for 1.8 million deaths (18.4% of the total) in 2018. A large-scale study demonstrated that up to 15% of lung cancer patients have two or more tumors [1]. Multiple tumors may represent intrapulmonary metastasis with a common origin, recurrence of a previously diagnosed tumor, or multiple primary lung cancers (MPLCs) of independent lineage. The distinction between intrapulmonary metastasis and multiple primaries is paramount for accurate staging as ‘multiple primaries’ can be ranged from T1-T4 while ‘intrapulmonary metastasis’ is at least a T3 disease. The best treatment plan for patients with MPLCs essentially depends on accurate discrimination between multiple primaries and intrapulmonary metastasis. To date, many surgeons, oncologists, and pathologists agree that neither current clinical guidelines nor any market available methodologies can provide precise information for making this distinction.

Several novel approaches have been developed to assist in the diagnosis of MPLC. However, no significant improvements have been yielded so far. The methodologies of these inventions are based on advanced platforms, ranging from biomarker detection, computerized tomography (CT) scan, and next-generation sequencing. The clinical utilities of these systems are still limited by low detection sensitivity and a stringent requirement on sample input. Although the clonality test for immunoglobulin and T-cell receptors is well-established for lymphoid malignancies, this technique is not applicable in other solid cancers. Because misclassification of a patient's tumor status scarifies the patient's opportunity to receive the appropriate treatment plan, improved MLPC detection and tumor classification methods are needed.

### BRIEF SUMMARY OF THE INVENTION

The instant invention provides methods and systems for differentiating multiple primary cancers from metastases in an organ of a cancer patient. The metastasis in a said organ can originate from cancer outside said organ or from cancer originating from the same organ. The methods and systems of the invention quantify common clonality between two or more tissue samples obtained from an organ of a subject having or suspected to have cancer. Based on the methods and systems of the instant invention, tissue samples originating from metastases in the organ are characterized by common clonality. In contrast, tissue samples not originating from metastases are featured by a lack of common clonality. Advantageously, by analyzing and quantifying the copy number variation features of at least two specimens from an organ of a patient and taking the whole genomic structure at a low resolution into account, the methods and systems of the instant invention provide differentiation of multiple primary cancers from metastases in said organ with very high accuracy and at lower cost than currently used methods.

### BRIEF DESCRIPTION OF THE DRAWINGS

FIG. 1 shows a log₂ transformed copy number for a paired sample analyzed using the instant methods in which scoring bins have a discordant focal copy number variation pattern (see label (a)), a log₂ transformed copy number for a sample analyzed using the instant methods in which scoring bins do not have a focal copy number variation (see label (b)), and a log₂ transformed copy number for non-scoring bins analyzed using the instant methods (see label (c)).

FIG. 2 shows the optimization of MVP parameters. The box plot shows the change of MVP scores after the optimization step listed in each stage listed.

FIGS. 3A and 3B show the overall performance of MVP in the discovery and validation cohort. FIG. 3A shows the usage of the simulated MPLC dataset to derive 6, which corresponds to a type 1 error equal to 1%. The distribution of MVP scores across the MPLC and MLC samples and its corresponding confusion matrix. FIG. 3B shows the performance of a, R & 6 to classify MPLC and MLC in the validation cohort.

### DETAILED DISCLOSURE OF THE INVENTION

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. In the context of reagent and/or analyte concentrations, the term “about” can mean a range of up to 0-20%, 0 to 10%, 0 to 5%, or up to 1% of a given value. In the context of pH measurements, the terms “about” or “approximately” permit a variation of ±0.1 unit from a stated value.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned; for example, a range of 5-10 indicates all the values between 5.0 and 10.0 as well as between 5.00 and 10.00, including the terminal values.

The term “biological sample” or “sample from a subject” encompasses a variety of sample types obtained from an organism. The term encompasses bodily fluids such as blood, blood components, saliva, nasal mucous, serum, plasma, cerebrospinal fluid (CSF), urine and other liquid samples of biological origin, solid tissue biopsy, solid tumors, tissue cultures, or supernatant taken from cultured patient cells. In the context of the present disclosure, the biological sample is typically a bodily fluid with detectable amounts of a subject's genome, e.g., a tissue sample, blood or a blood component (e.g., plasma or serum), saliva, oropharyngeal, nasopharyngeal, or a nasal secretion (mucous). The biological sample can be processed prior to assay, e.g., to remove cells or cellular debris. The term encompasses samples that have been manipulated after their procurement, such as by treatment with reagents, solubilization, sedimentation, or enrichment for specific components.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “isolated nucleic acid” molecule refers to a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose or polyacrylamide) containing restriction-digested genomic DNA, is not an “isolated nucleic acid”.

The term “genome” generally refers to the entirety of an organism's hereditary information. A genome can be encoded either in DNA or RNA. A genome can comprise coding regions that code for proteins as well as non-coding regions. A genome can include the sequence of all chromosomes together in an organism. For example, the human genome has 23 pairs of chromosomes. The sequence of all of these together constitutes a human genome.

The terms “genomic bin(s)” and “bin(s)” as used herein refer to a continuous region of an intact chromosome which is fixed-sized in length and without overlapped regions with adjacent bins. The bin size could vary from 1 kilobase (kb) to 1000 kilobase. In addition, each genomic bin has a unique identity representing its genomic location (chromosome number, starting position, and ending position). Under this scenario, chromosome 1 in the human genome is 248387328 base pair in length, divided into 250 genomic bins of 1000 kb in size. In addition, the identities of the first and the second 1000 kb-genomic bin of chromosome 1 are chr1:1-1000000 and chr1:1000001-2000000, respectively.

The term “copy number” as used herein refers to the total copy number per uniquely identified genomic bin of each chromosome present within a single cell. For instance, the copy number of all uniquely identified genomic bins in each chromosome in a human body cell/somatic cell is equal to two since each cell has a pair of each chromosome, one is the paternal origin, and another one is the maternal origin.

The term “tumor cellularity” as used herein refers to the portion of cancer cells present in a tumor excision. In addition to cancer cells, other tumor-infiltrating cells include cells derived from the immunological system, blood vessels, and connective tissue. Tumor cellularity is a crucial factor influencing nucleic acid quality for molecular diagnosis. For instance, a somatic copy number variation with one copy gain present in the tumor cell-associated nucleic acid may not be detectable if the tumor cellularity is lower than 5%.

The term “copy number variation” and “somatic copy number variation (SCNV)” as used herein refers to any designed change in the copy number of the genomic bin(s) in a tumor cell. A somatic copy number variation is regarded as amplification or gain if the acquired copy number of a bin or a set of contiguous bins is greater than the copy number amplification constant. On the other hand, when compared to the copy number in a normal cell, a copy number variation is regarded as deletion or loss if the acquired copy number of a bin or a set of contiguous bins is smaller than the copy number deletion constant. Moreover, SCNV is classified as a chromosomal or a chromosome-arm level event if all bins of a single chromosome or all bins of an arm of a chromosome are simultaneously amplified or deleted, respectively. Focal SCNV is referred to all types of copy number variation in a genomic region with sizes less than 3M base pairs.

The term “copy number amplification constant” as used herein refers to an experiment-derived numeric parameter. Any genomic bin with a copy number value greater than the copy number amplification constant is regarded as a copy number gain or copy number amplified region. The experiment deriving this constant is the whole genome sequencing of a set of known standards, i.e., a group of metastatic and multiple primary origin tumors.

The term “copy number deletion constant” as used herein refers to an experiment-derived numeric parameter. Any genomic bin with a copy number value smaller than the copy number deletion constant is regarded as a copy number gain or copy number amplified region. The experiment deriving this constant is the whole genome sequencing of a set of known standards, i.e., a group of metastatic tumors and multiple primary origin tumors.

The term “breakpoint” as used herein refers to a genomic position in which two adjacent genomic bins of a chromosome possess different copy numbers regardless of the copy number amplification/deletion constant. For instance, a breakpoint is present in chr1:1000000 if the copy number of the first and the second 1000-kb bin are 2 and 3, respectively.

The terms “clonality” and “clonal relationship” as used herein refer to a biological pheromone in which a group of cells arose by multiple rounds of cell division of one single ancestor cell. All daughter cells share identical genetic material. Thus, only one genotype could be found in a group of cells with the same clonality. For instance, all body cells, except terminally differentiated immune cells and germ cells, in any animal are given rise by multiple rounds of cell division of a fertilized egg. Therefore, all body cells, except terminally differentiated immune cells and germ cells, in one animal share the same clonality. In the same vein, all body cells in monozygotic twins share the same clonality as they are developed from the same fertilized egg. In tumor biology, tumor cells share the same clonality when developed or evolved from the same tumor initiation cell. For example, lung adenocarcinoma tumor cells share different clonality with tumor cells of a brain tumor since they arose from different tumor initiation cells. However, after metastatic lung cancer spread into the brain, the tumor cells isolated from the lung (primary site) and the brain (metastatic site) share the same clonality since they are developed/evolved from the same tumor initiation cell.

Provided are systems and methods for differentiating multiple primary cancers (MPCs) from metastases in an organ of a patient suffering from cancer, which multiple primary cancers are characterized by a lack of common clonality whereas said metastatic cancers are characterized by common clonality, whereby the methods and systems of the invention quantify common clonality by measuring the degree of similarity in somatic copy number variation (SCNV) in at least two tissue specimen obtained from a said organ.

Specifically, the methods and systems of the invention comprise the following steps: 1) preparation of nucleic acids from at least two tissue samples obtained from an organ of a subject and identified as being cancerous and low pass whole genome sequencing (LPWGS) of the nucleic acids, 2) bioinformatics pipelines for analyzing the next-generation sequencing (NGS) data, and 3) treatment of the subject with a therapeutic composition that treats a primary cancer of the organ if the subject suffers from MPCs and treating the subject with a therapeutic composition that treats metastases if the subject suffers from metastases in said organ.

In some embodiments of the invention, systems and methods are provided for differentiating multiple primary cancers in an organ (MPCs) from metastases in the organ where MPCs have a low degree of common clonality and metastases that can originate either from non-organ cancer or an organ cancer have a high degree of common clonality, where the methods and systems of the invention quantify the degree of similarity in somatic copy number variation (SCNV) shared by two or more tissue specimens obtained from the organ of a subject having cancer or suspected to have cancer in the organ and wherein a clinician identifies the tissue specimens as being cancerous tissue specimens (e.g., Sample A and Sample B).

In some embodiments, the tissue samples obtained from the organ of a subject having cancer or suspected to have cancer in the organ are identified as being cancerous tissue specimens by methods including, but not limited to, histochemistry, immunohistochemistry, electron microscopy, flow cytometry, image cytometry, cytogenetics, fluorescent in situ hybridization, polymerase chain reaction, gene expression microarray, and DNA sequencing.

In some embodiments, the tissue samples are submitted to the analyses and quantifications of the instant invention without any prior identification of tumor regions in the tissue samples. In certain embodiments, if only a very small percentage of the submitted tissue is cancer, there is a need for tumor region identification, optionally followed by microdissection.

In specific embodiments of the invention, systems and methods are provided for differentiating multiple primary lung cancers (MPLCs) from lung metastases where MPLCs have a low degree of common clonality and metastases that originate either from non-lung cancer or lung cancer have a high degree of common clonality, where the methods and systems of the invention quantify the degree of similarity in somatic copy number variation (SCNV) shared by two or more tissue specimens obtained from the lungs of a subject having cancer or suspected to have cancer in the lung and identified as being cancerous tissue specimens (e.g., Sample A and Sample B).

In specific embodiments of the invention, systems and methods are provided for differentiating multiple primary liver cancers (MPLiCs) from liver metastases where MPLiCs have a low degree of common clonality and metastases that originate either from non-liver cancer, or liver cancer have a high degree of common clonality, where the methods and systems of the invention quantify the degree of similarity in somatic copy number variation (SCNV) shared by two or more tissue specimens obtained from the liver of a subject having cancer or suspected to have cancer in the liver and identified as being cancerous tissue specimens.

In specific embodiments of the invention, systems and methods are provided for differentiating multiple primary kidney cancers (MPKCs) from kidney metastases where MPKCs have a low degree of common clonality and metastases that originate either from non-kidney cancer or a kidney cancer have a high degree of common clonality, where the methods and systems of the invention quantify the degree of similarity in somatic copy number variation (SCNV) shared by two or more tissue specimens obtained from the kidneys of a subject having cancer or suspected to have cancer in the kidneys and identified as being cancerous tissue specimens.

In specific embodiments of the invention, systems and methods are provided for differentiating multiple primary gastric cancers (MPGCs) from gastric metastases where MPGCs have a low degree of common clonality and metastases that originate either from a non-gastric cancer or a gastric cancer have a high degree of common clonality, where the methods and systems of the invention quantify the degree of similarity in somatic copy number variation (SCNV) shared by two or more tissue specimens obtained from the stomach of a subject having cancer or suspected to have cancer in the stomach and identified as being cancerous tissue specimens.

In specific embodiments of the invention, systems and methods are provided for differentiating multiple primary breast cancers (MPBCs) from gastric metastases where MPBCs have a low degree of common clonality and metastases that originate either from a non-breast cancer or a breast cancer have a high degree of common clonality, where the methods and systems of the invention quantify the degree of similarity in somatic copy number variation (SCNV) shared by two or more tissue specimens obtained from the breast of a subject having cancer or suspected to have cancer in the breast and identified as being cancerous tissue specimens.

In yet further embodiments, the system and methods of the instant invention can be used to differentiate multiple primary cancers (MPCs) from metastases wherein the primary cancers and the cancers from which the metastases originate can be any of hepatocellular carcinoma, bile duct cancer, bladder cancer, bone cancer, brain cancer, breast cancer, bronchial cancer, carcinoid tumor, cardiac tumor, cervical cancer, colon cancer, colorectal cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, head and neck cancer, heart cancer, hypopharyngeal cancer, pancreatic cancer, adrenocortical carcinoma, Kaposi sarcoma, teratoid/rhabdoid tumor, basal cell carcinoma, laryngeal cancer, lip and oral cavity cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

In some embodiments, the methods of the invention comprise the preparation of nucleic acid for low pass whole genome sequencing. In some embodiments, the tissue samples or biopsies or excision specimens obtained from an organ of a subject having or suspected to have cancer, before analysis and quantification according to the methods and systems of the instant invention, are examined by methods including, but not limited to, histochemistry, immunohistochemistry, electron microscopy, flow cytometry, image cytometry, cytogenetics, fluorescent in situ hybridization, polymerase chain reaction, gene expression microarray, and DNA sequencing. In some embodiments, the tissue samples are submitted to be subjected to analysis and quantification according to the methods and systems of the instant invention without any prior identification of tumor regions in the tissue samples.

In some embodiments, the tissue samples are micro-dissected for deoxyribonucleic acid (DNA) extraction. The extracted DNA is mechanically sheared to 200 base pairs (bp) in length. After quantification, the DNA fragments are converted to a next-generation sequencing-ready library and sequenced using low pass whole genome sequencing (LPWGS) to a goal genomic coverage of 1X.

In some embodiments, all raw sequencing data are collected, quality checked, and the raw sequencing reads passing quality control are aligned to a human reference genome build. In some embodiments, PCR duplications are marked and removed. Subsequently, the human genome-aligned sequencing data are divided into non-overlapping fixed-sized bins. The size of the genomic bins can be from about 1 to about 1000 kilobase, 1 to about 900 kilobase, 1 to about 850 kilobase, 1 to about 800 kilobase, 1 to about 750 kilobase, 1 to about 700 kilobase, 1 to about 650 kilobase, 1 to about 600 kilobase, 1 to about 550 kilobase, 1 to about 500 kilobase, 1 to about 450 kilobase, 1 to about 400 kilobase, 1 to about 350 kilobase, 1 to about 300 kilobase, 1 to about 250 kilobase, 1 to about 200 kilobase, 1 to about 150 kilobase, 1 to about 100 kilobase, or 1 to about 50 kilobase.

In some embodiments, for each bin, the log₂-transformed copy number (log₂B) is calculated with a simultaneous correction for sequencing bias, GC-content, mappability, and tumor cellularity.

In specific embodiments of the invention, the sequencing data of all bins of a pair of tissue samples are categorized and scored. All scores are quantified to arrive at a Metastasis Versus Primary (MVP) score.

In some embodiments, numerical MVP scores are calculated for each paired sample of tissue specimens obtained from a subject according to a specific MVP scoring algorithm. A numerical MVP score is calculated for each paired tissue sample by performing the following steps: i) normalizing the tumor cellularity, ii) filtering and categorizing genomic bins, iii) adding breakpoint adjustment, and iv) comparing and quantifying the log 2 transformed copy number on a genome-wide scale. Advantageously, the design of the MVP score offers a simple readout of the similarity between the samples in the tested tissue pair.

In some embodiments, the log₂ transformed copy number of all bins that arose from a sample is normalized according to corresponding tumor cellularity. This adjusts the pre-analytical error caused by inter-tumoral variation in the percentage of the tumor-infiltrating cells, including cells derived from the immune system, cells derived from the blood vessel, and connective tissue. Without a proper normalization step, a true SCNV would be mis-classified as a background noise. This affects both the sensitivity and specificity of MVP score. The normalization system is conducted according to the following formula:

$$z ( CNVi ) = \frac{CNVi - \frac{1}{T B} {\sum }_{TB}^{1} CNV}{\sqrt{\frac{{Σ ( CNVi - μ )}^{2}}{TB}}}$$

In specific embodiments, the genomic bins are categorized and scored according to the tumor cellularity-adjusted copy number of a first sample and the tumor cellularity-adjusted copy number of a second sample at a genomic bin location, wherein the genomic bin location copy numbers are scored according to a copy number amplification constant and copy number deletion constant. The thresholds of the copy number amplification constant and copy number deletion constant can be obtained by calibration process of laboratories equipment and workflow, when on average, at least one copy gain/loss of at least half of the bin region can be reliably detected. The copy number amplification constant and copy number deletion constant are slightly larger than the theoretical value to accommodate background noise.

In some embodiments, a genomic bin is categorized as a scoring bin in a first and a second sample obtained from an organ of a subject having or suspected to suffer from cancer when the tumor cellularity-adjusted copy numbers of the first sample and the second sample at the same genomic bin are above the copy number amplification constant.

In some embodiments, a genomic bin is categorized as a scoring bin in a first and a second sample obtained from an organ of a subject having or suspected to suffer from cancer when tumor cellularity-adjusted copy numbers of the first sample and the second sample at the same genomic bin are below the copy number deletion constant.

In specific embodiments, a genomic bin is categorized as a non-scoring bin when the tumor cellularity-adjusted copy number at the genomic bin location is below the copy number amplification constant and above the copy number deletion constant.

In specific embodiments, a genomic bin is categorized as a non-scoring bin when genomic segments in the same bin are not amplified or deleted in the first and the second tissue sample. According to the invention, they are, therefore, defined as having tumor cellularity-adjusted transformed copy numbers of both the first sample and the second sample that are larger than a copy number deletion constant and smaller than a copy number amplification constant. In this invention, these bins are non-informative.

According to the methods and systems of the invention, focal copy number variations include any designed change in the copy number of the genomic bin(s) in a tumor cell. A focal copy number variation is regarded as amplification or gain if the acquired copy number of a bin or a set of contiguous bins is greater than the copy number amplification constant.

Breakpoints quantified by the methods and systems of the instant invention are conserved in tumor samples of the same clone and are relatively unaffected by tumor cellularity. Therefore, differences in breakpoint patterns represent a reduced resemblance between clonality of tumor samples of a pair and, therefore, are suggestive of multiple primary cancers.

In specific embodiments, the measurement of a breakpoint according to the methods and systems of the invention represents a unique characteristic of a sample obtained from an organ of a subject having cancer or suspected to have cancer. If a second sample obtained from the organ does not comprise a similarly located breakpoint, such as, for example, within two genomic bin locations, of the breakpoint bin location of the first sample, this difference in pattern is suggestive of multiple primary cancers.

In some embodiments, the score of a bin of a particular genomic location bin is adjusted by a breakpoint coefficient (φ), where the breakpoint coefficient for a specific bin (φi) is calculated according to the formula:

(φi)=Aⁿⁱ

wherein A is a pre-defined constant between 0 and 1, and ni is the number of breakpoints unique to only one of a first and a second sample in the genomic region covered by the segment that includes the bin at the specified genomic location (i), whichever number of breakpoints is larger.

In some embodiments, each bin is scored according to Si, wherein Si is a score of a bin at a genomic location (i) and wherein

Si=0 if a bin at the genomic location (i) does not belong to a sample with copy number amplifications and/or deletions, i.e. non-scoring bin, and
Si=C×(φi) if a bin at the genomic location (i) belongs to a sample with copy number amplifications and/or deletions, i.e. scoring bin;
wherein C is a pre-defined positive constant>1.

In some embodiments, an MVP score is calculated to represent the degree of SCNV pattern similarity between two tumor tissue. It is a ratio of the total number of the scoring bin(s) to a fraction of the non-scoring bin. The MVP score is calculated according to the following formula:

$${MVP}_{score} = \frac{{\sum }_{i = 1}^{TB} {S}_{i}}{1 - \frac{number of {B}_{nor}}{TB}}$$

In embodiments of the invention, a high MVP score indicates a higher degree of similarity in somatic copy number variation and, therefore, a clonal relationship between the samples, and a low MVP score indicates a lower degree of similarity in somatic copy number variation and, therefore, no clonal relationship between the samples.

Accordingly, metastases have a high MVP score quantified using the methods and systems of the invention, and multiple primary cancers have a low MVP score.

Further, tumor sample pairs with many scoring bins have a high MVP score and are obtained from metastases of an organ of a subject. Tumor sample pairs with many non-scoring bins have a low MVP score and are obtained from multiple primary cancers of an organ of a subject.

In some embodiments, an MVP score cut-off S is defined by a one-tailed test of a simulated MPLC dataset, where a pair of tissue samples from an organ of a subject having cancer or suspected to have cancer with an MVP score larger than or equal to δ is considered as being of metastatic origin. In contrast, a pair of samples from an organ of a subject having cancer or suspected to have cancer with an MVP score smaller than δ is considered as being of multiple primary origins.

In some embodiments, a pair of tissue samples are from a lung of a subject having cancer or suspected of having cancer, the MVP score is quantified to a value higher than a S cut-off value, and the samples are clonally related and thus from metastases.

In some embodiments, a pair of tissue samples are from a lung of a subject having cancer or suspected of having cancer, the MVP score is quantified to a value lower than a S cut-off value, and the samples are from multiple primary cancers.

During the process of tumorigenesis, a tumor cell acquires a unique SCNV pattern through a series of chromosome amplifications and deletions. These SCNV patterns are only shared by tumors originating from the same tumor-initiating cells and are characterized by the same clonality. In contrast, tumors that originate from different original tumor cells have different SCNV and are not characterized by clonality. The instant invention provides systems and methods to measure, analyze, summarize, and quantify this biological feature of unique SCNV patterns of clonal tumors as Metastasis Versus multiple Primary score (MVP score).

Advantageously, the instant methods and systems provide MVP scores with excellent accuracy for the diagnosing MPLC and proper treatment of a patient suffering from cancer that is an MPLC or constitutes a metastasis from non-lung cancer. Compared to other diagnostic approaches, the instant methods and systems provide a superior diagnostic value at a lower cost and higher applicability to a wider spectrum of sample types. Therefore, the instant invention provides systems and methods for routine clinical tests used in MPLC diagnosis.

Advantageously, the instant invention provides data processing and manipulation of CNV data generated compared to only CNV detection. Further, the instant invention determines the clonality (i.e. relationship) between two tissue samples from a patient instead of a characteristic of any one sample. It establishes cancer clonality with a different approach than prior methods by comparing the CNV features of two tissue specimens taking whole genomic structure at a low resolution into account. Thereby, the instant invention provides differentiation of, e.g., double lung adenocarcinoma from metastasis with very high accuracy and at a lower cost than currently used clinical methods.

Advantageously, the methods and systems of the instant invention offer the highest detection sensitivity among all MPLC diagnostic methods. In addition, the scoring system of the instant invention is well designed to provide user-friendly and instructive information to clinicians, providing an opportunity to choose an appropriate therapy for cancer patients that is geared towards, e.g., treating a primary lung adenocarcinoma using therapeutic compositions that are known to be useful in treating a primary lung adenocarcinoma and treating lung metastases using therapeutic compositions that are known to be useful to treat cancer from which the metastases originate or are known to treat metastases.

Further advantageously, the methods and systems of the invention employ a combination of low pass whole genome sequencing with specific categorizing and scoring of sequence bins and summarizing log 2 transformed copy number in a genome-wide scale such that readout data on tumor sample originality from metastases or multiple primary cancers can readily and at low cost be provided to the clinical setting.

The treatments according to the instant invention can include any treatment modality that is available to a clinic to treat cancer including, but not limited to, therapeutic antibodies, kinases, alkylating agents, platinum-based agents, intercalating agents, antibiotics, inhibitors of mitosis, taxanes, inhibitors of topoisomerase and antimetabolites and include all-trans retinoic acid, azacitide, azathioprine, bleomycin, carboplatin, capecitabine, cisplatin, chlorambucil, cyclophosphamide, cytarabine, daunorubicin, docetaxel, doxifluridine, doxorubicin, epirubicin, epothilone, fluorouracil, gemcitabine, hydroxyurea, idarubicin, imatinib, mechlorethamine, mercaptopurine, methotrexate, mitoxantrone, oxaliplatin, paclitaxel, pemetrexed, teniposide, tioguanine, trofosfamide, valrubicin, vinblastine, vincristine, vindesine, and vinorelbine. Advantageously, the methods and systems of the instant invention are compatible with all current clinically used human perseveration methods and require only a sample input of as low as 10 ng of genomic DNA.

In certain embodiments, the subject provides methods for differentiating multiple primary cancers from metastases in a subject and treating the subject. The method comprises:

(i) extracting a sample of nucleic acids from at least two tissue samples of a subject diagnosed with cancer, wherein the samples comprise deoxyribonucleic acid (DNA) molecules;

(ii) sequencing the DNA molecules from at least two tissue samples and receiving a plurality of sequence reads;

(iii) executing a plurality of instructions using a computer product comprising a non-transitory computer-readable medium, wherein the plurality of instructions control a computer system to derive a diagnostic factor from a log₂ transformed copy number of the DNA molecules for the at least two tissue samples extracted from the subject. The plurality of instructions for determining the log₂ transformed copy number of the DNA comprises:
a. aligning the plurality of sequence reads derived from the sample to a reference genome; and
b. dividing the reference genome into a fixed number of non-overlapping genomic bins; and
c. calculating the log₂ transformed copy number of each genomic bin;
d. normalizing the log₂ transformed copy numbers in all genomic bins of the first tissue sample and the second tissue sample according to the tumor cellularity;
   wherein normalizing the log₂ transformed copy numbers of all genomic bins is performed according to the formula:

$$z ( CNVi ) = \frac{CNVi - \frac{1}{TB} {\sum }_{TB}^{1} CNV}{\sqrt{\frac{{Σ ( CNVi - μ )}^{2}}{TB}}}$$

wherein CNV(i) is the apparent log₂ copy number change in any genomic bin; TB is the total number of non-overlapping genomic bins, and z(CNVi) is the z-score transformed log₂ copy number in genomic bin i;
e. categorizing a genomic bin as a scoring bin (Sᵢ) if the adjusted log₂ transformed copy number after step (e) from both the first sample and the second sample are greater or equal to the copy number amplification constant, or less than the copy number deletion constant;
f. categorizing a genomic bin as a non-scoring bin (Bₙₒᵣ) if the adjusted log₂ transformed copy number after step (e) from the first sample and the second sample are both in-between the copy number amplification constant and the copy number deletion constant;
g. identifying the breakpoint(s) present in a continuous series of scoring bins and adjusting the scored bins with a breakpoint coefficient (y) (y), wherein the breakpoint coefficient is performed according to the following formula:
   (φᵢ)=Aⁿⁱ, wherein A is a pre-defined constant between 0 and 1, and n is a number of breakpoints unique to only one of the first tissue sample and the second tissue sample in the genomic region covered by the segment at the specified genomic location (i);
h. determining a clonal relationship of the first and the second tissue sample using the adjusted normalized log₂ of transformed copy numbers, according to the following formula:

$${MVP}_{score} = \frac{{\sum }_{i = 1}^{TB} {S}_{i}}{1 - \frac{number of {B}_{nor}}{TB}}$$

wherein TB is the total number of genomic bins; and
i. assigning the first and the second tissue sample as metastases if the score obtained in step h) is greater than or equal to a validated constant or assigning the first and the second tissue samples as multiple primary cancers if the score obtained in step h) is less than the validated constant; and

(iv) treating the subject with a therapeutic composition known to be effective against cancer from which the metastases originate if the tissue samples are assigned as metastases or treating the subject with a therapeutic composition known to be effective against multiple primary cancers if the tissue samples are assigned as multiple primary cancers.

All patents, patent applications, provisional applications, and publications referred to or cited herein are incorporated by reference in their entirety, including all figures and tables, to the extent they are not inconsistent with the explicit teachings of this specification. Following are examples that illustrate procedures for practicing the invention. These examples should not be construed as limiting. All percentages are by weight, and all solvent mixture proportions are by volume unless otherwise noted.

### MATERIALS AND METHODS

### Example 1—Differentiation of a Double Primary Lung Adenocarcinoma from Metastatic Lung Cancer

Systems and methods of the instant invention are used to differentiate, e.g., a double primary lung adenocarcinoma from metastatic lung cancer by investigating the degree of similarity in somatic copy number variation shared by more than one biopsy or excision specimens (e.g., Sample A and Sample B). The method comprises 1) preparation of nucleic acid and low pass whole genome sequencing (LPWGS) and 2) bioinformatic analysis of the sequencing data.

### Example 2—Preparation of Nucleic Acid for Low Pass Whole Genome Sequencing

Pathologists examine lung biopsies or excision specimens to identify tumor regions that are subsequently micro-dissected for deoxyribonucleic acid (DNA) extraction. The extracted DNA is mechanically sheared to 200 base pairs (bp) in length by a focused-ultrasonicator (Covaris®). After DNA quantification by Qubit (Invitrogen®), the DNA fragments are converted to next generation sequencing (NGS) library by Kapa Hyper Prep kit (Kapa®). To complete LPWGS, the DNA library is sequenced at 1X genomic coverage by Illumina®-based next-generation sequencing.

### Example 3—Bioinformatics Analysis of the Sequencing Data

The NGS data are converted to a precise scoring system for guiding MPLC diagnosis. All raw sequencing data are collected in fastq format and quality checked by FastQC 0.11.9. The raw sequencing read passing quality control is aligned to human reference genome build GRCH37/hg19 with BWA 0.7.17. PCR duplications are marked and removed by Picard 2.21.9. Then, the human genome is divided into non-overlapping fixed-sized bins. The log₂-transformed copy number of each bin (log₂B) is calculated by qDNAseq 1.22.0, with correction for sequencing bias, GC-content, and mappability.

### Example 4—Mvp Scoring of Samples

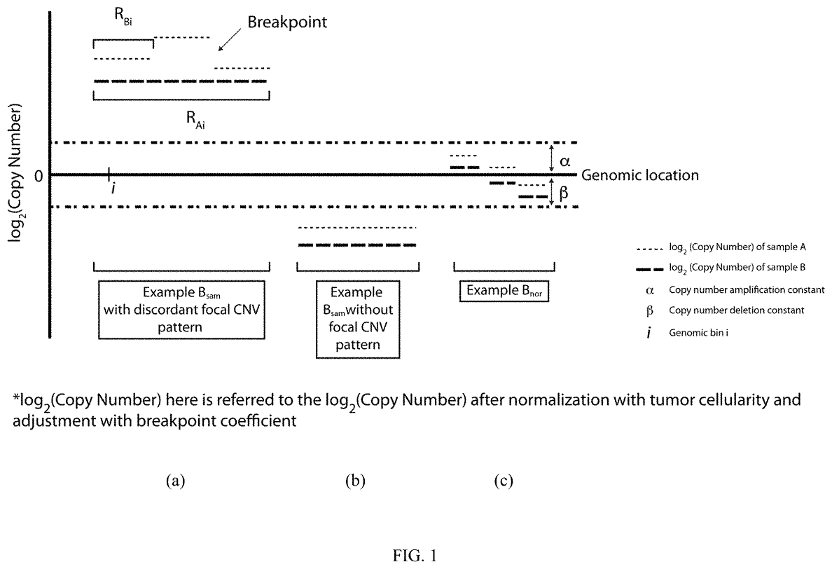

A scoring algorithm developed by the inventors, named Metastasis Vs. Primary (MVP) calculates a numerical score (MVP score) for each paired sample by: i) normalizing tumor cellularity, ii) filtering and categorizing genomic bins, iii) adding breakpoint adjustment and iv) comparing and summarizing the log₂ transformed copy number in a genome-wide scale. The MVP score is designed to enable a simple readout that signifies the similarity between the samples in the tested tissue pair (see FIG. 1)

### Example 5—Categorizing and Scoring of the Genomic Bins as Shown in FIG. 1

TABLE 1

| Classes | Meaning of the class | definition |
|---|---|---|
| Bₙₒᵣ | Not amplified or | β < log₂B_{A} & log₂B_{B} < α |
| (non-scoring bin) | deleted in both samples | |
| Bₛₐₘ | amplified in both | log₂B_{A} & log₂B_{B} > α |
| (scoring bin) | samples or deleted | or |
| | in both samples | log₂A & log₂B < β |

log₂B_{A} & log₂B_{B}: log₂ transformed copy number of sample A and sample B
α: An in-house derived threshold, any log₂B higher than it is considered as a copy number amplified region
β: An in-house derived threshold, any log₂B lower than it is regarded as a copy number deleted region.

ii) Adjustment for Focal copy number variations

### Example 6—Measurement of Focal Copy Number Variations (Cnvs)

Whenever there is a focal CNV event, breakpoints are formed in the copy number profile plot when two adjacent genomic bins of the same chromosome have a different log₂-transformed copy number (log₂B). Because the breakpoints are conserved in tumors of the same clonality and are relatively unaffected by tumor content, differences in breakpoint patterns indicate a reduced resemblance of a sample pair.

A breakpoint is unique to one sample if the other sample does not show a breakpoint within two adjacent genomic bins. To account for the subtle focal CNV events, the score of Bsam bins is adjusted by a breakpoint coefficient (φ). The breakpoint coefficient (φ) is calculated for each bin of every genomic location (i), and the following formula defines it:

Breakpoint coefficient(φi)=Aⁿⁱ

A: a predefined constant between 0 and 1.
nᵢ: number of breakpoints unique to only 1 of the samples in R_{Ai} or R_{Bi}, whichever is larger.
R_{Ai}: genomic region covered by the segment that includes bin at genomic location i in sample A
R_{Bi}: genomic region covered by the segment that includes bin at genomic location i in sample B

### Scoring of Each Bin

Sᵢ=C×φᵢ if a bin at genomic location i belongs to Bₛₐₘ, i.e., scoring bin
Sᵢ=0 if a bin at genomic location i does not belong to Bₛₐₘ, i.e., a non-scoring bin
C: a pre-defined positive constant>1
Sᵢ: score of the bin at genomic location i

### Example 7—Summarizing Data into an Mvp Score

Scores of all bins are summed, and they are divided by the percentage of non-Bₙₒᵣ bins.

$${MVP}_{score} = \frac{{\sum }_{i = 1}^{TB} {S}_{i}}{1 - \frac{number of {B}_{nor}}{TB}}$$

TB: total number of genomic bins

A higher Metastasis Vs Primary (MVP) score signifies a high degree of similarity in somatic copy number variation, thus, suggesting a clonal relationship between the samples.

### Example 8—Validation

An MVP_{score} cut-off value, δ, was defined using one-tailed test of simulated MPLC dataset. A pair of samples with an MVP_{score} larger than δ was considered as a metastatic origin. In contrast, a pair of biopsies with an MVP_{score} smaller than δ was considered as multiple primary origins.

Although there is currently no gold standard for differentiating double primary lung cancers from metastatic diseases, the performance of the MVP score was compared to targeted sequencing results that covered over 166 cancer genes. Total of 42 pairs of adenocarcinomas from 38 patients were confirmed either metastatic or multiple primary origins by targeted sequencing. Among them, 34 pairs (81%) were concordant with clinical-pathological information. Classification of these samples using the instant invention was fully concordant in the 34 classifiable pairs.

### Example 9—Comparison of the Instant Invention to Clinical-Pathological Diagnosis

The instant invention uses methods to perform a genome-wide detection, analysis, and comparison of CNV signature from lung excision or biopsy specimens and provides an unbiased standard with high sensitivity and accuracy for MPLC diagnosis in clinical use.

Current clinical guidelines for diagnosing MPLC, the American Joint Commission on Cancer staging manual (AJCC 8th edition), and the College of American Pathologists (CAP) are mainly based on clinical-pathological parameters [3, 4]. For patients presenting with multiple lung tumors at the same time, MPLC is only classified if discordant histology is present, i.e., adenocarcinoma vs squamous carcinoma, or tumors with concordant histology are anatomically distinct or have differences in composition of the predominant cellular subtype and/or cytological features. For patients with recurrent lung tumors, in addition to the criteria of different histology, MPLC is diagnosed if there is a four years or greater interval between the two tumors.

These clinical-pathological parameters have limitations in the diagnosis of MPLC. The clinical guidelines are not clearly defined. Even when all clinical parameters are precisely investigated, equivocal or incongruous diagnoses are frequently drawn among different pathologists [5-8]. In addition, the median interval between a primary and a recurrent lung cancer is two years. Thus, the four-year interval requirement of the current clinical guideline potentially leads to misclassification of MPLC cases. The instant invention clearly outperforms a clinical-pathological diagnosis by offering a more precise definition of diagnostic criteria and applying to a broader range of recurrent cases.

### Example 10—Comparison of the Instant Invention to Biomarker-Based Diagnosis

The use of different biomarkers to distinguish MPLC against intrapulmonary metastasis has been proposed for many years. These methods include but are not limited to immunohistochemical staining of lineage markers (TTF1, p40, p63 & NapsinA) [9], analysis of DNA microsatellites [10], mutation status evaluation of single genes such as TP53, KRAS, PTEN and EGFR [11-15], or targeted sequencing of a gene panel [16]. All these methods share a common problem; they suffer from a low detection sensitivity. Since the mutation pattern of lung cancer may vary significantly, neither a single biomarker nor a group of biomarkers can recapitulate all somatic mutations. For instance, a driver mutation is observed in 30% of lung adenocarcinoma data [17], making a biomarker-based test only applicable to 70% of cases. High mutation rates of some common hotspots, e.g., EGFR L858R and exon 19 deletions, make the interpretation challenging because the odds of co-occurrence by chance can be high. Consequently, there is no commercial product based on biomarkers on the market to distinguish MPLC against intrapulmonary metastasis.

In contrast, the instant invention offers higher sensitivity. The diagnosis is made according to a comprehensive evaluation of copy number variations on a genome-wide scale. Thus, the methods and systems of the instant invention possess more data points than any currently used methods. With the additional implementation of a breakpoint coefficient, the instant invention significantly increases the sensitivity without compromising specificity.

### Example 11—Comparison of the Instant Invention to Array-Based Comparative Genomic Hybridization (ACGH)

It has been proposed that aCGH can compare the copy number variation between multiple tissue specimens [1, 18]. However, this method requires a relatively large amount of sample DNA, limiting its usefulness in small specimens, especially biopsies. In comparison, the instant invention requires much smaller amounts of input DNA, e.g., 10 ng in LPWGS compared to 1 μg in aCGH. Moreover, the resolution of aCGH is dependent on the number of probes available on a chipset. Thus, to achieve a resolution of CNV detection as provided by the instant invention, the cost of an aCGH chipset is significantly higher. Therefore, compared to aCGH, the instant invention has higher marketability because it requires less DNA input and can be performed at a lower price.

### Example 12—Comparison of the Instant Invention to Ct Scans

A retrospective study developed an algorithm using computed tomography (CT) to conduct MPLC diagnosis [19]. The overall accuracy for classifying clinical confirmed MPLC was up to 89%. However, this algorithm required experienced radiologists to analyze CT images and was affected by inter-observer variations [8, 20-23]. Moreover, it required supportive facilities and skilled radiologists to deploy the diagnostic service. The instant invention achieves 100% diagnostic accuracy on all retrospective cases as documents using targeted sequencing but requires no radiology facilities or skilled radiologists.

### Example 13—Comparison of the Instant Invention to an Identification of Genome-Wide Breakpoints

Mate-pair sequencing has been used to detect chromosome breakpoints and diagnose MPLCs [24]. The methods and systems of the instant invention not only offer better diagnostic accuracy but also outperform the chromosome breakpoint approach by higher applicability to a broader range of specimens, a faster turn-over time, and lower sequencing costs. Mate-pair sequencing requires DNA extraction from fresh or frozen tissue, which limits its use in conventional clinical environments. Mate-pair sequencing cannot use with archived tissue, so it is impossible to determine recurrence. Moreover, the tissue needs to be micro-dissected by laser capture microdissection, which extends the processing procedure and increases the difficulty of DNA sampling and costs. To ensure the validity of the breakpoint, a sequencing depth of the mate-pair sequencing approach is at least 40 times higher than the instant invention is required. Therefore, the methods and systems of the instant invention provide superior performance with broader applicability at lower cost and with less involvement of clinical specialists.

### Example 14—Implementation, Parameter Selection, and Validation of the Results

A sample implementation protocol has been established to demonstrate the present system's diagnostic capability. It involves identifying a cohort with established tumor status, (multiple primary lung cancer (MPLC) vs metastatic lung cancer (MLC)), tuning all parameters, and validating by using a dataset.

### 1) Patients and Samples

Non-small cell lung cancer (NSCLC) patients who received surgical resection at Prince of Wales Hospital, Hong Kong, between 1996 and 2018 were selected for model building. The study protocol was approved by the Joint CUHK-NTE Clinical Research Ethics Committee. The patient inclusion criteria include:

i. The patient presented with multiple tumors, either synchronous or metachronous cases;

ii. The patient has no known diagnosis of other cancer;

iii. Patients are not treated with any neoadjuvant chemotherapy or radiotherapy before surgery.

After the preliminary selection based on these criteria, the formalin-fixed paraffin-embedded (FFPE) surgical resections were reviewed by pathologists to confirm and grade the tumor based on definitions in the eighth edition of the American Joint Committee on Cancer (AJCC) Tumor, Node, Metastasis (TNM) staging system (2017). Two independent retrospective cohorts, a discovery cohort, and a validation cohort, in total consisting of 199 tumor specimens from 86 patients have been included in this study. The discovery and validation cohorts are tumor tissue isolated from lung cancer patients with multiple cancer sites/histories. The samples are separately processed to do LPWGS in two independent batches. The first and second batches were completed in 2017 and 2020, respectively. This is a necessary procedure to ensure the observations obtained in any cohort were not caused by any systemic or random error that arose from a single experiment. On the other hand, we use these two independent cohorts to validate reproducibility.

### 2) Dataset

All cases were reviewed by a scientific officer and an experienced pathologist with an interest in lung pathology. The status of each tumor pair was determined by a combination of clinicopathological data and mutation signatures. Clinical information includes patient records, imaging results, and follow-up data. Pathological information are pathology report and the slides of the cancer tissue submitted for testing at the time of diagnosis. The mutation signature of each sample was generated by targeted sequencing comprising a 166-gene panel. The analysis considered tumor pairs as metastatic origin, i.e. MLC, if they harbor the same type of mutation on key driver genes such as EGFR, KRAS, ALK, MET, ROS1, RET, NRAS, ERBB2, BRAF, NF1, AKT1, PIK3CA, and FGFR1. Tumors were classified as MPLC if they had completely different mutation signatures. Tumor pairs with no known mutations in the 166 selected genes or discordant classification from targeted sequencing and clinicopathological data were excluded from the study. In the end, we got 46 tumor tissue pairs with known disease statuses. These tumors are used as the gold standard, which can be a created set of data with known answers, particularly a dataset with a combination of clinical pathological and molecular data create a dataset with known answers, such as, for example, metastatic vs. multiple primaries, for us to establish and calibrate the MVP algorithm. We further randomly divided these cases into a discovery cohort (n=26 cases) for tuning all parameters, and a validation cohort (n=20 cases), for validating MVP performance.

By assuming tumors from two patients are biologically irrelevant, we systematically paired all inter-patient tumors, irrespective of disease status, to create a dataset mimicking MPLC conditions. This resulted in a simulated MPLC cohort. We further randomly divided these simulated MPLC pairs into a discovery cohort (n=4485 simulated pairs) and a validation cohort (n=9336 simulated pairs). The causes of tumorigenesis in different patients can be different and thus not share the same clonality. By this assumption, we treated tumors from two patients as simulated MPLC samples.

### 3) Optimization of MVP Parameters

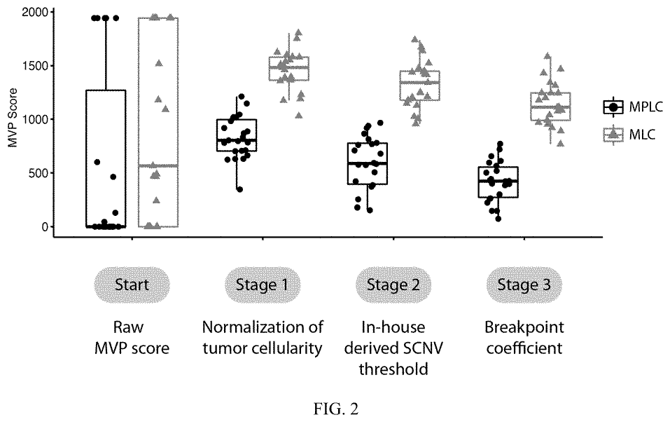

An essential step in the implementation stage was the optimization of all key parameters in the MVP algorithm (FIG. 2). Each optimization step improved MVP classification power by correcting technical challenges in corresponding aspects.

A. Genomic Bin Size

Genomic bin size is a key parameter to control the resolution when comparing the SCNV patterns. The smaller the bin size, the higher the sensitivity to detect small changes in SCNV patterns, at the cost of compromised specificity. These SCNV patterns of all included samples were evaluated by using two publicly available NGS pipelines, ichorCNA, and qDNAseq, yielding a chain of log₂ transformed copy numbers across the genome, representing the SCNV at different regions of the genome. We tested the bin size ranging from 50 kb to 1000 kb. With consideration of sequencing depth, the measured standard deviations (representing noise in qDNASeq, which is an inbuilt performance metric of the qDNASeq package, as described by Scheinin I, Sie D, Bengtsson H, van de Wiel M A, Olshen A B, van Thuijl H F, van Essen H F, Eijk P P, Rustenburg F, Meijer G A, Reijneveld J C, Wesseling P, Pinkel D, Albertson D G, Ylstra B. DNA copy number analysis of fresh and formalin-fixed specimens by shallow whole-genome sequencing with identification and exclusion of problematic regions in the genome assembly. Genome Res. 2014 December; 24(12):2022-32, which is hereby incorporated by reference in its entirety), fragment number and fluctuation of the log 2 transformed copy number, bin size of 1000 kb performed best. After analyzing the performance metrics in the discovery cohort and SCNV plots, the bin size was determined.

B. Selection of the Tumor Content Normalization Method

Lung tumor excision specimens have a varying percentage of normal cell and cancer content. A variation in tumor content will produce different log₂ transformed copy numbers even for the same SCNV event. For instance, the log 2 transformed copy number of a two-copy number chromosomal amplification is theoretically equal to 2, which changes to 1.58 and 1.13 in tumors with 50% and 10% tumor cellularity, respectively. All qDNAseq-derived log 2 transformed copy numbers must be normalized with the tumor content to improve accuracy. Error in the normalization process induces discordant regions among otherwise concordant regions, falsely favoring MPLC over MLC.

NGS algorithm ichorCNA estimates the effect of variation in tumor cellularity, and NGS algorithm CNVkit corrects the effect of variation in tumor cellularity. We tested both algorithms with our discovery cohort. Although the algorithms functioned as designed in discovery cohort cases, the reproducibility of the derived parameters was unsatisfactory. The classification accuracy dropped below 90% in the validation cohort (data not shown).

Changes in tumor cellularity systematically affect the log₂ transformed copy number of all SCNV in a sample to the same extent. The following formula could interpret the qDNAseq-derived log 2 transformed copy number of any genomic bins:

CNV(i)=log 2(2×TC+CN×(1−TC))

CNV(i): the apparent log₂ copy number change in any genomic bin
TC: Tumor cellularity in a range of 0 to 1

CN: The actual copy number change in any genomic bin We took advantage of this biological phenomenon to develop an in-house normalization method. We first fixed the sequencing depth to be 10 million reads per sample, assuming tumor cellularity and the presence of SCNV are the only factors influencing log₂ transformed copy number across two samples. In this way, we hypothesize that the z-score of all log₂ values in a pair of MLP tumor samples with different tumor cellularity should be close in terms of value. Alternatively, a significant difference in term of value will be noticed in the z-score of all log₂ transformed copy numbers in a pair of MPLC samples. Therefore, we applied a z-score transformation to all qDNAseq-derived log₂ transformed copy numbers, which is being used for all subsequent calculations.

$$z ( CNVi ) = \frac{CNVi - \frac{1}{T B} {\sum }_{TB}^{1} CNV}{\sqrt{\frac{{Σ ( CNVi - μ )}^{2}}{TB}}}$$

CNV(i): the apparent log₂ copy number change in any genomic bin
Z(CNVi): the z-score transformed log₂ copy number in genomic bin i.
TB is the total number of non-overlapping genomic bins.

C. Cutoff for Amplification (α)/Deletion (β) and MVP Score (δ)

The intrinsic nature of the low coverage whole genome sequencing led to fluctuations of the log₂ transformed copy number in copy-neutral genomic regions, even after tumor content normalization. A cutoff to separate a true SCNV event from background noise is thus necessary for MVP analysis. The cutoff for amplification (α)/deletion (β) constant can be obtained by calibration process of laboratories equipment and workflow, when at least on average one copy gain/loss of at least half of the bin region can reliably be detected. For fine-tuning, different combinations of amplification (α)/deletion (β) cut-offs around ±0.3 have been used in the calculation of MVP in the discovery cohort, and the performance of different combinations was benchmarked by the successful classification of MPLC and MLC groups.

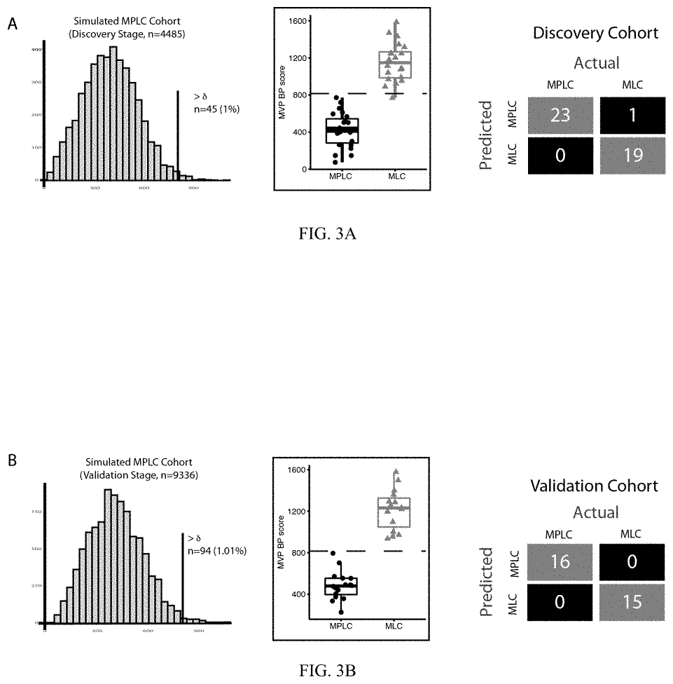

The MVP score (δ) cutoff was determined by using the simulated MPLC dataset derived from the discovery cohort. It was chosen at the 99^{th} percentile of the MVP scores of the cohort mentioned above, which represented a 1% chance of misdiagnosing an MPLC case as an MLC case (FIG. 3A).

D. Breakpoint Coefficient

Recurrent SCNV patterns have been identified in many cancer types; lung cancer is not an exceptional case. Without proper handling, MPLC tumors harboring a high portion of recurrent SCNV regions would be misclassified as MLP. We invented a breakpoint coefficient to distinguish a recurrent SNCV from concordant SCNV. It detected and applied a negative weighting to all breakpoints inside recurrent SCNV. By minimizing the influence of recurrent SCNV, MVP increases the capability to resolve border cases, i.e., cases with MVP scores close to the cutoff (814). The base of the Breakpoint coefficient (A) was chosen to be 0.7.

### 4) Validation

Parameters used in this implantation (both discovery and validation cohort) are listed as follows in Table 2:

TABLE 2

| Package for generating | |
|---|---|
| SCNV from NGS data | qDNAseq |
| Normalization method | Self-developed |
| | z-score based method |
| Cut off for amplification (α) | 0.38 |
| Cut off for deletion (β) | 0.38 |
| Base of the Breakpoint coefficient (A) | 0.7 |
| Cut off for MVP score (δ) | 814 |
| Bin score for Bₛₐₘ (C) | 2* |
| Genomic bin size | 1000 kb (kilo basepair) |

*Any positive constant >1 will work. Changing (C) will affect the scale of the MVP score but not the differentiating power.

### 5) Results

After fixing 1000 kb as the genomic bin size and developing the tumor cellularity normalization method, the remaining parameters, including the threshold for amplification/deletion, breakpoint coefficient, and MVP score (i.e., α, β, A, and δ), were successfully optimized. In the discovery cohort, the classification accuracy reached the best result when the cutoff for both amplification (α)/deletion (β) were both 0.38. Based on these parameters, the cutoff for the MVP score (δ) was determined to be 814. At this cutoff, the MVP algorithm separated MPLC from MLC in the discovery cohort with 100% sensitivity and 95% specificity (FIG. 3A).

The model and parameters were validated using the simulated MPLC validation cohort and the validation cohort. In the simulated MPLC validation cohort, the cutoff for the MVP score (δ) corresponded to the 98.99th percentile of the MVP scores of the cohort mentioned above, which represented a 1.01% chance of misdiagnosing an MPLC as MLC (FIG. 3B). With the actual validation cohort, we achieved 100% sensitivity and specificity.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Girard, N., et al., Genomic and mutational profiling to assess clonal relationships between multiple non-small cell lung cancers. Clin Cancer Res, 2009. 15(16): p. 5184-90.
2. Martini, N. and M. R. Melamed, Multiple primary lung cancers. J Thorac Cardiovasc Surg, 1975. 70(4): p. 606-12.
3. Kozower, B. D., et al., Special treatment issues in non-small cell lung cancer: Diagnosis and management of lung cancer, 3rd ed: American College of Chest Physicians evidence-based clinical practice guidelines. Chest, 2013. 143(5 Suppl): p. e369S-e399S.
4. Loukeri, A. A., et al., Metachronous and synchronous primary lung cancers: diagnostic aspects, surgical treatment, and prognosis. Clin Lung Cancer, 2015. 16(1): p. 15-23.
5. Homer, R. J., Pathologists' staging of multiple foci of lung cancer: poor concordance in absence of dramatic histologic or molecular differences. Am J Clin Pathol, 2015. 143(5): p. 701-6.
6. Chen, C., et al., Multiple primary lung cancer: a rising challenge. J Thorac Dis, 2019. 11(Suppl4): p. S523-S536.
7. Marchevsky, A. M., Problems in pathologic staging of lung cancer. Arch Pathol Lab Med, 2006. 130(3): p. 292-302.
8. Nicholson, A. G., et al., Interobserver Variation among Pathologists and Refinement of Criteria in Distinguishing Separate Primary Tumors from Intrapulmonary Metastases in Lung. J Thorac Oncol, 2018. 13(2): p. 205-217.
9. Ye, J., et al., Combination of napsin A and TTF-I immunohistochemistry helps in differentiating primary lung adenocarcinoma from metastatic carcinoma in the lung. Appl Immunohistochem Mol Morphol, 2011. 19(4): p. 313-7.
10. Shen, C., et al., Microsatellite alteration in multiple primary lung cancer. J Thorac Dis, 2014. 6(10): p. 1499-505.
11. Mitsudomi, T., et al., Mutations of the P53 tumor suppressor gene as clonal marker for multiple primary lung cancers. J Thorac Cardiovasc Surg, 1997. 114(3): p. 354-60.
12. Chang, Y L., et al., Clonality and prognostic implications of p53 and epidermal growth factor receptor somatic aberrations in multiple primary lung cancers. Clin Cancer Res, 2007. 13(1): p. 52-8.
13. Yang, Y, et al., Phenotype-genotype correlation in multiple primary lung cancer patients in China. Sci Rep, 2016. 6: p. 36177.
14. Liu, M., et al., Surgical treatment of synchronous multiple primary lung cancers: a retrospective analysis of 122 patients. J Thorac Dis, 2016. 8(6): p. 1197-204.
15. Zhu, Z., T. Yu, and Y Chai, Multiple primary lung cancer displaying different EGFR and PTEN molecular profiles. Oncotarget, 2016. 7(49): p. 81969-81971.
16. Roepman, P., et al., Added Value of 50-Gene Panel Sequencing to Distinguish Multiple Primary Lung Cancers from Pulmonary Metastases: A Systematic Investigation. J Mol Diagn, 2018. 20(4): p. 436-445.
17. Yeung, S. F., et al., Profiling of Oncogenic Driver Events in Lung Adenocarcinoma Revealed MET Mutation as Independent Prognostic Factor. J Thorac Oncol, 2015. 10(9): p. 1292-1300.
18. Vincenten, J. P. L., et al., Clonality analysis of pulmonary tumors by genome-wide copy number profiling. PLoS One, 2019. 14(10): p. e0223827.
19. Suh, Y J., et al., A Novel Algorithm to Differentiate Between Multiple Primary Lung Cancers and Intrapulmonary Metastasis in Multiple Lung Cancers With Multiple Pulmonary Sites of Involvement. J Thorac Oncol, 2020. 15(2): p. 203-215.
20. Al-Khawari, H., et al., Inter- and intraobserver variation between radiologists in the detection of abnormal parenchymal lung changes on high-resolution computed tomography. Ann Saudi Med, 2010. 30(2): p. 129-33.
21. Louie, A. V., et al., Inter-observer and intra-observer reliability for lung cancer target volume delineation in the 4D-CT era. Radiother Oncol, 2010. 95(2): p. 166-71.
22. Persson, G. F., et al., Interobserver delineation variation in lung tumour stereotactic body radiotherapy. Br J Radiol, 2012. 85(1017): p. e654-60.
23. McErlean, A., et al., Intra- and interobserver variability in CT measurements in oncology. Radiology, 2013. 269(2): p. 451-9.
24. Murphy, S. J., et al., Using Genomics to Differentiate Multiple Primaries From Metastatic Lung Cancer. J Thorac Oncol, 2019. 14(9): p. 1567-1582.
25. Murphy, S. J., et al., Identification of independent primary tumors and intrapulmonary metastases using DNA rearrangements in non-small-cell lung cancer. J Clin Oncol, 2014. 32(36): p. 4050-8.
26. Detterbeck, F. C., et al., The IASLC Lung Cancer Staging Project: Background Data and Proposed Criteria to Distinguish Separate Primary Lung Cancers from Metastatic Foci in Patients with Two Lung Tumors in the Forthcoming Eighth Edition of the TNM Classification for Lung Cancer. J Thorac Oncol, 2016. 11(5): p. 651-665.
27. Lam, S., C. MacAulay, and B. Palcic, Detection and localization of early lung cancer by imaging techniques. Chest, 1993. 103(1 Suppl): p. 12S-14S.
28. Ferguson, M. K., et al., Diagnosis and management of synchronous lung cancers. J Thorac Cardiovasc Surg, 1985. 89(3): p. 378-85.
29. Woolner, L. B., et al., Roentgenographically occult lung cancer: pathologic findings and frequency of multicentricity during a 10-year period. Mayo Clin Proc, 1984. 59(7): p. 453-66.
30. Verhagen, A. F., et al., Surgical treatment of multiple primary lung cancers. Thorac Cardiovasc Surg, 1989. 37(2): p. 107-11.
31. van Rens, M. T., et al., Survival in synchronous vs. single lung cancer: upstaging better reflects prognosis. Chest, 2000. 118(4): p. 952-8.
32. ADDIN EN.REFLIST ADDIN EN.REFLIST Amin M B, EdRge S, Greene F, Byrd D R, Brookland R K, Washington M K, Gershenwald J E, Compton C C, Hess K R, et al. (Eds.). AJCC Cancer Staging Manual (8th edition). Springer International Publishing: American Joint Commission on Cancer; 2017.

We claim:

1. A method for differentiating multiple primary cancers from metastases in a subject and treating the subject, the method comprising:
i) extracting a sample of nucleic acids from at least two tissue samples of a subject diagnosed with cancer, wherein the samples comprise deoxyribonucleic acid (DNA) molecules;
ii) sequencing the DNA molecules from at least two tissue samples and receiving a plurality of sequence reads;
iii) executing a plurality of instructions using a computer product comprising a non-transitory computer-readable medium, wherein the plurality of instructions controls a computer system to derive a diagnostic parameter from a log 2 transformed copy number of the DNA molecules for at least two tissue samples extracted from the subject, the plurality of instructions for determining the copy number-derived diagnostic parameter comprises:
a) aligning the plurality of sequence reads derived from the samples to a reference genome;
b) dividing the reference genome into non-overlapping genomic bins with size fixed;
c) calculating the log₂ transformed copy number of each genomic bin for all samples;
d) normalizing the log₂ transformed copy numbers in all genomic bins of the first tissue sample and the second tissue sample according to the corresponding tumor cellularity;
e) categorizing a genomic bin as a scoring bin (Sᵢ) if the tumor cellularity-adjusted copy number after step (e) from both the first sample and the second sample are greater or equal to a copy number amplification constant, or less than a copy number deletion constant;
f) categorizing a genomic bin as a non-scoring bin (Bₙₒᵣ) if the tumor cellularity-adjusted copy number from the first sample and the second sample are both in-between the copy number amplification constant and the copy number deletion constant;
g) identifying the breakpoint(s) present in a continuous series of scoring bins and adjusting the scored bins with a breakpoint coefficient (p);
h) determining a clonal relationship of the first and the second tissue sample using the MVP scoring formula; and
i) assigning the first and the second tissue sample as metastases if the score obtained in step h) is greater than or equal to a validated constant or assigning the first and the second tissue samples as multiple primary cancers if the score obtained in step h) is less than the validated constant; and
iv) treating the subject with a therapeutic composition known to be effective against cancer from which the metastases originate if the tissue samples are assigned as metastases or treating the subject with a therapeutic composition known to be effective against the multiple primary cancers if the tissue samples are assigned as multiple primary cancers.

2. The method according to claim 1, further comprising scoring a tumor cellularity-adjusted copy number of the same genomic bin(s) of the at least two samples as a copy number amplified region when the log₂ copy numbers are higher than the copy number amplification constant.

3. The method according to claim 1, further comprising scoring a tumor cellularity-adjusted copy number copy number of the same genomic bin(s) of the at least two samples as a copy number deleted region when the log₂ copy numbers lower than the copy number deletion constant.

4. The method according to claim 1, wherein tumor cellularity-adjusted transformed copy numbers in the same genomic location are scoring bins when the bin segments from two samples are simultaneously higher or lower than the copy number amplification constant or copy number deletion constant, respectively.

5. The method according to claim 1, wherein tumor cellularity-adjusted transformed copy numbers in the same genomic location from the two samples are discordant when the bin from only one of the paired samples is higher (or lower) than the copy number amplification constant (or copy number deletion constant), or when bin from one sample is higher than the copy number amplification constant while the corresponding bin from another sample is lower than the copy number deletion constant.

6. The method according to claim 1, wherein a therapeutic composition known to be effective against a cancer from which the metastases originate comprises a therapeutic antibody, a kinase, an alkylating agent, a platinum-based agent, an intercalating agent, an antibiotic, an inhibitor of mitosis, a taxane, an inhibitor of a topoisomerase, or an antimetabolite.

7. The method according to claim 1, wherein a therapeutic composition known to be effective against multiple primary cancers comprises a therapeutic antibody, a kinase, an alkylating agent, a platinum-based agent, an intercalating agent, an antibiotic, an inhibitor of mitosis, a taxane, an inhibitor of a topoisomerase, or an antimetabolite.

8. The method of claim 1, wherein the breakpoint coefficient (y) of a particular genomic bin (i) is calculated according to the formula:
(φi)=Aⁿⁱ
wherein A is a predefined constant between 0 and 1, and n is a number of breakpoints unique to only one of the first tissue sample and the second tissue sample in the genomic region covered by the segment at the specified genomic location (i).

9. The method of claim 8, wherein the number of breakpoints is the larger number of breakpoints from the first tissue sample or the second tissue sample.

10. The method of claim 1, wherein normalizing the log₂ transformed copy numbers of all genomic bins is performed according to the formula:
$$z ( CNVi ) = \frac{CNVi - \frac{1}{T B} {\sum }_{TB}^{1} CNV}{\sqrt{\frac{{Σ ( CNVi - μ )}^{2}}{TB}}}$$
wherein CNV(i) is the apparent log₂ copy number change in any genomic bin; TB is the total number of non-overlapping genomic bins; and z(CNVi) is the z-score transformed log₂ copy number in genomic bin i.

11. The method of claim 1, wherein the clonal relationship is determined according to the following formula:
$${MVP}_{score} = \frac{{\sum }_{i = 1}^{TB} {S}_{i}}{1 - \frac{number of {B}_{nor}}{TB}}$$
wherein TB is the total number of genomic bins.
